(19) United States
(12) Patent Application Publication
(10) Pub. No.: US 2008/0020053 A1
(43) Pub. Date: Jan. 24, 2008

(54) Solid Dosage Form Comprising Proton Pump Inhibitor and Suspension Made Thereof

(75) Inventors: Eva Persson, Lund (SE); Eva Trofast, Lund (SE)

Correspondence Address: WHITE & CASE LLP;PATENT DEPARTMENT, 1155 AVENUE OF THE AMERICAS, NEW YORK, NY, 10036, US

(73) Assignee: ASTRAZENECA AB, Sodertalje (SE)

(21) Appl. No.: 11/722,387

(22) PCT Filed: Dec. 20, 2005
(86) PCT No.: PCT/SE05/01972

§ 371 Date: Jun. 21, 2007

Related U.S. Application Data
(60) Provisional application No. 60/638,435, filed on Dec. 22, 2004.

(51) Int. Cl. A61K 31/4439; A61K 31/444; A61K 47/36; A61K 9/14; A61P 43/00

(52) U.S. Cl. 424/490; 514/303; 514/338; 514/777

(57) ABSTRACT

A solid rapidly gelling oral pharmaceutical dosage form, as well as aqueous suspensions prepared thereof, comprising an acid sensitive proton pump inhibitor as active ingredient distributed in a multitude of enteric coated pellets and a suspension modifying granulate comprising a rapidly dissolving diluent granulated together with a gelling agent chosen among xanthan gums, and an acidic pH-regulating agent and a binder. The suspension modifying granulate is rapidly disintegrating and gelling when suspended in an aqueous medium and thus forming a homogenous stable and robust suspension having a reproducible and stable viscosity. Furthermore the invention relates to an improved process for its manufacture and the use of such formulation in medical treatment including prevention of gastrointestinal disorders in humans.

### FIELD OF THE INVENTION

This invention relates to a solid rapidly gelling oral pharmaceutical dosage form, as well as aqueous suspensions prepared thereof, comprising an acid sensitive proton pump inhibitor as active ingredient distributed in a multitude of enteric coated pellets and a suspension modifying granulate. Furthermore the invention relates to an improved process for its manufacture and the use of such formulation in medical treatment including prevention of gastrointestinal disorders in humans.

### BACKGROUND OF THE INVENTION AND PRIOR ART

Proton pump inhibitor (in the following also designated as “PPI”) compounds having effect as H⁺K⁺-ATPase inhibitors are for instance compounds known under the generic names omeprazole, lansoprazole, pantoprazole, rabeprazole, tenatoprazole and esomeprazole.

These active substances are useful for inhibiting gastric acid secretion in mammals and man. In a more general sense, they may be used for prevention and treatment of gastric acid related diseases in mammals and man, including e.g. reflux esophagitis, gastritis, duodenitis, gastric ulcer and duodenal ulcer. Furthermore, they may be used for treatment of other gastrointestinal disorders where gastric acid inhibitory effect is desirable e.g. in patients on NSAID therapy, in patients with Non Ulcer Dyspepsia, in patients with symptomatic gastro-esophageal reflux disease, and in patients with gastrinomas. They may also be used in patients in intensive care situations, in patients with acute upper gastrointestinal bleeding, pre- and postoperatively to prevent acid aspiration of gastric acid and to prevent and treat stress ulceration. Further, they may be useful for prevention and treatment of irritable bowel syndrome (IBS), inflammatory bowel disease (IBD), ulcerative colitis, Crohn's disease, asthma, laryngitis, Barret's syndrome, sleep apnea, sleep disturbance, psoriasis as well as being useful for prevention and treatment of Helicobacter infections and diseases related to the above.

These active compounds are, however, susceptible to degradation/transformation in acidic and neutral media. The degradation is catalyzed by acidic compounds and is stabilized in mixtures with alkaline compounds. The stability of the active substances is also affected by moisture, heat, content of organic solvents and to some degree by light.

Oral dosage forms remain a significant problem for many patients, as many are unable or unwilling to swallow a solid dosage form. This problem occurs primarily in children and the elderly. It affects the patient compliance, and is therefore a problem in therapy.

The need for an oral administration form, which avoids the swallowing difficulties associated with traditional tablets, has been recognized since many years. Syrups, elixirs, microcapsules containing slurries and other novel tablet or capsule dosage forms have been developed. Among alternative forms for oral administration of pharmacologically active substances is the use of a solution or a suspension of the active ingredient in an aqueous medium.

Besides that ready to consume suspensions (or solutions) have drawbacks associated with larger storage volume and often limited shelf-life or need for refrigerator storage, a particular problem that sometimes arises with aqueous suspensions is that some solid particles have a strong tendency of sinking to the bottom of the vessel used for administration. This may cause a part of the dose to be retained in the vessel and not the entire dose entering the oral administration route. Another problem that is sometimes experienced, is when using a suspension of particles in a liquid medium for administration through a nasogastric tube, the particles may tend to aggregate or agglomerate, thereby making it impossible for them to pass through the used tube. Still another problem is when the liquid medium has a too high viscosity/viscoelasticity, which is making it impossible to administer it through a nasogastric tube at a practical pressure.

It is a strong desire, particularly when administering acid-labile compounds like proton pump inhibitors such as for instance omeprazole, esomeprazole, pantoprazole and lansoprazole, to get an easily and quickly prepared, easily swallowable homogeneous suspension comprising the proton pump inhibitor in a form protecting it from contact with acidic environments, (e.g. the acidic gastric fluids). In addition it is desirable that the suspension has viscoelastic properties and a viscosity suitable for allowing it to be administered via a gastric tube or to be swallowed. Also a liquid suspension formulation requires a certain viscosity to be stable over time.

With a drug preparation that is to be stored as a dry powder mixture comprising water unsoluble components, and which is intended to be given as an extempore prepared homogeneous suspension, other challenges/problems arise.

For some prior art compositions there is a problem with that a maximum viscosity level is obtained only after long times, i.e. the viscosity is not constant over the short time frames from which the suspension is made until it usually is administered to the patient. There may also be problems with batch-to-batch variation regarding time required to obtain a stable maximum viscosity level in the suspension prepared from a dry powder mixture.

Intolerance to lactose-containing foods is a common problem. Thus, medicaments containing lactose may pose a problem for such people.

There are proposals in the art regarding compositions comprising a proton pump inhibitor and there are other proposals relating to methods for quickly dispersing and/or dissolving formulations.

U.S. Pat. No. 5,731,002 describes a stable, oral pharmaceutical composition comprising a proton pump inhibitor in a paste-like gel designed for the treatment of gastric acid related diseases in animals.

U.S. Pat. No. 5,840,737 discloses a method for treating gastric acid disorders with compositions comprising omeprazole or lansoprazole together with bicarbonates.

Problems associated with administering bicarbonates such as sodium- or potassium bicarbonate to i.a. humans include that when the carbonate is neutralized in the stomach belching may result. Patient with gastroesophageal reflux may exacerbate or worsen their disease as the belching can cause upward movement of stomach acid (Brunton, Agents for the control of gastric acidity and treatment of peptic ulcers. In: Goodman A G, et al. The pharmacologic basis of therapeutics, p. 907. (New York, 1990.)) Moreover, there is a possibility that intake of sodium bicarbonate may cause metabolic alkalosis.

Furthermore, there are further published patent applications in the same patent family, such US 2002/0045646 A1, which discloses a solid nor-enteric coated dosage form compositions comprising a proton pump inhibitor and a buffer. Other applications in the family US2003/118669, US2003/144306, US2003/191159, US2003/215527, US2004/048896 and US2004/171646 disclose for instance liquid oral pharmaceutical compositions comprising a proton pump inhibitor and a buffering agent and method of increasing absorption of the proton pump inhibitor.

US 2004/0005362 A1 (Taneja) and US 2004/0082618 A1 (Taneja) describe a pharmaceutical formulation comprising an acid labile drug coated with an enteric coating and a liquid vehicle of pH less than 6.0. Other published applications from the same inventor describes for instance a liquid vehicle of a viscosity sufficient to suspend microgranules comprising a PPI (US 2004/0081700 A1) or (US 2004/0006109 A1 and US 2004/0081671 A1) principally the same arrangement wherein the pH of the liquid vehicle is greater than 6.5.

WO 2004/004690 A1 (Taneja) discloses a liquid dosage form having enteric coated microgranules comprising an acid-labile drug and a liquid suspension having a pH less than 6.0 and a viscosity sufficient to suspend the microgranules. Carbonates or bicarbonates may be used in the dosage forms.

US 2004/6022854 A1 discloses an oral administration form for acid-labile active compounds wherein the auxiliaries are not suitable for formation of enteric layers (enteric coating). Prepared active compound units can be formulated into sachets, e.g together with lactose, or formulated together with carbonate containing excipients to an effervescent composition.

EP 1,232,746 describes a readily suspendible dry powder mixture composition comprising a gellant or thickener, comprising at least one xanthan gum having a specific particle size distribution, a filler, a wetting agent or surfactant, and a pharmacologically active substance.

U.S. Pat. No. 4,886,669 describes a water-dispersible tablet comprising a pharmaceutically active agent, at least one disintegrant and a swellable material. It is stated that the tablet disintegrates rapidly in water forming a homogeneous suspension of high viscosity that can easily be swallowed.

U.S. Pat. No. 5,008,117 relates to a method for preparing a quickly dispersing and dissolving formulation of thickening or suspending agents and other excipients, in which drug microcapsules are readily dispersible. Proton pump inhibitors are not mentioned.

EP 0491910 B1 discloses a solid pharmaceutical composition for addition to water to produce a suspension of a drug. The composition comprises a thickening or suspending agent, an acid, and a carbonate or bicarbonate.

U.S. Pat. No. 6,261,602 describes a granular composition useful as a pharmaceutical carrier which can be used for the preparation of pharmaceutical compositions that are capable of rapid suspension in water or aqueous media. The composition may be prepared by a process which comprises subjecting a mixture of a thickening agent and a disintegrating agent to wet granulation with an aqueous medium as wetting agent or dry granulation to make a granular product.

### BRIEF DESCRIPTION OF THE INVENTION

The present invention avoids the above discussed disadvantages with prior art compositions and presents a solution to the previous mentioned problems. It further provides a mean for making a drug vehicle, which is suitable for administration via a gastric tube, due to good viscosity and viscoelasticity properties of the obtained vehicle (suspension). For instance, in the sense that it is e.g. robust enough to provide approximately the same viscosity even if the used water amount varies within 50% to 150% of the prescribed amount.

The present invention relates to a solid rapidly gelling oral pharmaceutical dosage form comprising an acid sensitive proton pump inhibitor compound as active ingredient distributed in a multitude of enteric coated pellets and a suspension modifying granulate.

Furthermore, it has now surprisingly been found that it is advantageous to use a special composed granulate to mix with a multitude of enteric coated pellets comprising a proton pump inhibitor, which granulate when suspended in water, quickly and reproducible will create an aqueous vehicle having a desired pH, a desired, stable viscosity level and a satisfying viscoelasticity. This granulate is in the following also referred to as “suspension modifying granulate”. Furthermore, this granulate should be free from bicarbonate and carbonate salts. According to one embodiment of the invention, it is possible to make this granulate free from lactose, i.e. tolerable for people having intolerance to lactose.

The dosage forms of the invention render the quick formation of a viscous stable suspension possible. Prior to administration, the solid dry suspension modifying granulate and the enteric coated pellet are dissolved/suspended in an aqueous liquid, such as tap water providing a viscous liquid formulation for oral administration. When a dosage form of the invention is to be administered to the patient, it is important that the preparation will be dissolved/suspended as fast as possible and at the same time provides a homogeneous suspension with regard to the distribution of the solid particles containing the pharmacologically active ingredient. Therefore, the final liquid formulation should secure that practically all of the dose, even if the dose is comprised in a suspended, particulate form, is delivered into the oral cavity, i.e. the entry of the oral administration route, in a safe, reliable and reproducible way.

When the active ingredient is comprised in enteric coated pellets, it is necessary that the suspension medium has a pH that does not cause premature dissolution of the enteric coating layer of the pellets comprising the active ingredient. Also the administration through naso-gastric tubes puts demands on the final liquid formulation regarding such things as suitable and stable viscosity, viscoelastic properties and absence of agglomeration tendencies of the suspended particles.

A further feature is that the suspension is suitable for administration with thin tubes aimed for pediatric use. The expression gastric tube includes naso-gastric tubes as well as other tubes or syringes aimed for feeding a suspension or dispersion into the stomach of a patient.

The viscoelastic and viscosity properties become especially important as tubes used in pediatric treatment may have a narrow inner diameter and thereby being sensitive for liquids having unsuitable properties giving high back-pressures upon administration. One such example of a tube with narrow inner diameter is “Infant feeding tube, FT 1606/105 (CH/FG 6-2.0 mm outer diameter, 1.4 mm inner diameter), Pennine Healthcare.”

The dosage forms of the invention are gelling quicker in water at room temperature than prior art formulations to yield a homogeneous stable dispersion. Thus, they give a stable viscosity in shorter time than the prior art, and furthermore they are robust in respect of obtained viscosity properties.

In brief, the dosage forms of the invention comprise two principal components; a suspension modifying granulate and a multitude of enteric coated pellets comprising the active ingredient.

The suspension modifying granulate comprises;
a rapidly dissolving diluent
a gelling agent
an acidic pH-regulating agent
a binder and
an optional disintegrant, and
   furthermore, the granulate is free from bicarbonate salts and/or carbonate salts.

According to one feature, the above described suspension modifying granulate is free from lactose. This further advantage makes it suitable for people suffering from lactose-intolerance and they can be treated with embodiments of the invention.

One of the features of the invention is that the rapidly dissolving diluent is brought into close/intimate contact with the gelling agent. This does not only give a very rapid gelling time compared to the gelling agent per se, but also very quickly a stable gel. The selection of an accurately diluent, which also may function as a sweetener, is one embodiment of the invention.

According to one feature of the invention, the rapid disintegration and quick gelling to a stable and reproducible viscosity level when the suspension modifying granulate is suspended in water, is achieved by a special manufacturing process. According to this feature the process comprises that the gelling agent and diluent/sweetener are mixed and granulated together and thereafter dried to obtain a low moisture and/or solvent content.

Manufacture of the enteric coated pellets is described in the section “Detailed description of the invention”, but they can in general be manufactured according to directives in WO 9601624 A1, with consideration taken to the special desires of size. Furthermore, there is no need for any “overcoat” on the enteric coated pellets.

The present invention provide a safe and reliable dosage forms for administration of enteric coated pellets comprising acid-labile proton pump inhibitors such as omeprazole, esomeprazole, pantoprazole and lansoprazole dispersed in an aqueous liquid medium, and also through gastric tubes. This is especially suitable and advantageous in the treatment of geriatrics or pediatrics.

The compositions of the present invention also allow the incorporation of a wide range of dosage levels and additional agents like taste masking/improving agents and tonicity agents.

### BRIEF DESCRIPTION OF THE FIGURES

FIG. 1 shows viscosity versus time for an embodiment of the invention. (5 samples)

FIG. 2 shows viscosity versus time for a prior art embodiment (Lanzo™, 4 samples).

### DETAILED DESCRIPTION OF THE INVENTION

One aspect of the present invention is a dosage form being a mixture of one component (I) being a multitude of enteric coated pellets and another component (II) being a suspension modifying granulate, the mixture being dispensed in a container like e.g. a sachet. The mixture is rapidly disintegrating and gelling when suspended in an aqueous medium, such as tap water, thus forming a homogeneous stable and robust suspension haying a reproducible and stable viscosity, a suspension that can easily be swallowed or administered through e.g. a naso-gastric tube, by the patient. The liquid formulation ready to use is a further aspect of the present invention, i.e. it comprises three components, the two components mentioned above (I) and (II) and in addition the liquid medium (III).

The rapid gelling, i.e. the short gelling time obtained, of the present invention, can i.a. be seen as an effect on the time required before substantially all of the enteric coated pellets in the prepared suspension remain suspended in the liquid medium and not sinking to the bottom of the vessel (glass, beaker), used for its preparation. The gelling time required for embodiments of the invention is in general shorter than 3 minutes, and preferably less than 2 minutes, when tested as described in Example 5.

The dosage form is free from bicarbonate salts and/or carbonate salts. One embodiment of the invention is furthermore free from lactose. “Free from” means that no such compound is added in the formulation. Trace amounts present in and accompanying other raw materials used in the composition are not taken into account by this expression.

Enteric Coated Pellets

The enteric coated pellets comprising the active ingredient are manufactured with the outermost layer being the enteric coating layer. Such pellets can be manufactured according to methods known in the art, e.g. as described in WO 9601624 A1, taking into consideration the special desires on size of the pellets. Furthermore, there is no need for any “overcoat” on the prepared enteric coated pellets.

According to one aspect of the invention, the average diameter of the enteric coated pellets is 0.2-1.8 mm in diameter, preferably 0.4-1.0 mm in diameter and more preferably 0.5-0.8 mm in diameter.

In another aspect of the invention the enteric coated pellets are in the size range of 1.0-1.4 mm in diameter.

The enteric coated pellets are consisting of the following structural components;
a core material comprising the active ingredient,
an optional separating or subcoating layer, and
an enteric coating layer,
   but no additional coating layer on the enteric coating layer.
   Core Material.

Core material is manufactured by processes known in the art such as extrusion-spheronization, layering techniques such as powder- or solution/suspension layering, spray drying, balling, congealing techniques or spray congealing techniques.

The core material comprises the active ingredient and may also comprise seeds, binders, surfactants, fillers, disintegrating agents, alkaline additives or other pharmaceutically acceptable ingredients, alone or in mixtures.

Active Ingredient

The pharmaceutical formulations of the invention comprise an acid sensitive proton pump inhibitor or an alkaline salt thereof or a single enantiomer or an alkaline salt of its enantiomer as active ingredient. The single enantiomers, racemic mixtures (50% of each enantiomer) and unequal mixtures of the two enantiomers are suitable for the pharmaceutical formulation according to the present invention.

The active ingredient is being comprised optionally together with excipients, in small enteric coated pellets/beads.

Compounds/active ingredients of interest for the novel pharmaceutical compositions according to the present invention are compounds of the general formula I, an alkaline salt thereof, one of the single enantiomers thereof or an alkaline salt of one of the enantiomers 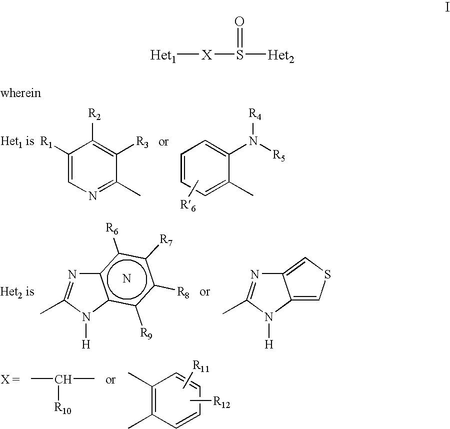
wherein
N in the benzimidazole moiety one that one of the ring carbon atoms substituted by R₆-R₉ optionally may be exchanged for a nitrogen atom without any substituents;
R₁, R₂ and R₃ are the same or different and selected from hydrogen, allyl, alkoxy optionally substituted by fluorine, alkylthio, alkoxyalkoxy, dialkylamino, piperidino, morpholino, halogen, phenyl and phenylalkoxy;
R₄ and R₅ are the same or different and selected from hydrogen, alkyl and arylalkyl;
R₆′ is hydrogen, halogen, trifluoromethyl, alkyl or alkoxy;
R₆-R₉ are the same or different and selected from hydrogen, alkyl, alkoxy, halogen, halo-alkoxy, alkylcarbonyl, alkoxycarbonyl, oxazolinyl, pyrrolyl and trifluoroalkyl, or adjacent groups R₆-R₉ form ring structures which may be further substituted;
R₁₀ is hydrogen or forms an alkylene chain together with R₃ and
R₁₁ and R₁₂ are the same or different and selected from hydrogen, halogen and alkyl.

In the above definitions alkyl groups, alkoxy groups and moities thereof may be branched or straight C₁-C₉-chains or comprise cyclic alkyl groups, for example cycloalkylalkyl.

Examples of specifically interesting compounds according to formula I are 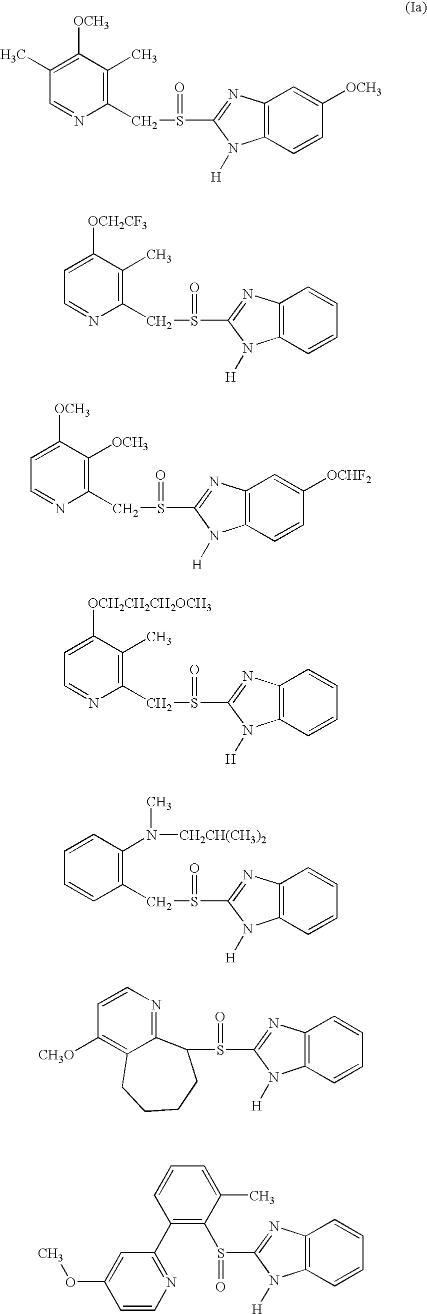 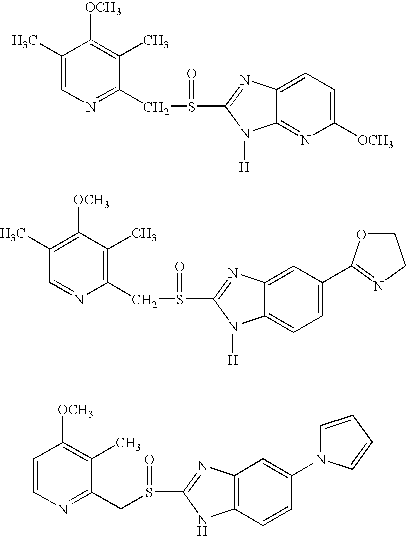
including tautomeric forms thereof.

Preferred compounds for the oral pharmaceutical preparation according to the present invention are omeprazole, a magnesium salt of omeprazole or a magnesium salt of the (−)-enantiomer of omeprazole. The latter having the generic name esomeprazole.

According to one embodiment the active ingredient is esomeprazole magnesium trihydrate. In another embodiment of the invention tenatoprazole or a pharmaceutically acceptable salt thereof, or a single enantiomer of either of them, is the active drug.

According to another aspect of the invention the compound/active ingredient is a hydrated form of anyone of the aforementioned compounds/active ingredients.

In one aspect of the invention, the amount of active ingredient in the preparation is in the range of 1 mg-100 mg, 2 mg-80 mg or 5 mg-50 mg.

Seeds

The seeds, which are to be layered with the active substance, can be water insoluble seeds comprising different oxides, celluloses, organic polymers and other materials, alone or in mixtures or water soluble seeds comprising different inorganic salts, sugars (excluding lactose), nonpareils and other materials, alone or in mixtures. Further, the seeds may comprise active substance in the form of agglomerates, compacts etc.

Binders

Binders are for example celluloses such as hydroxypropyl methylcellulose, hydroxypropyl cellulose and carboxymethyl-cellulose sodium, polyvinyl pyrrolidone, polyethylene glycols, polyvinyl alcohols, sugars (excluding lactose), starches and other pharmaceutically acceptable substances with cohesive properties.

Surfactants

Surfactants may be used in the dosage form. Suitable surfactants are found in the groups of pharmaceutically acceptable non-ionic surfactants such as for instance Polysorbate 80 or ionic surfactants such as for instance sodium lauryl sulfate.

Fillers

Fillers may be used in the dosage form Examples of fillers include for instance mannitol and dicalcium phosphate.

Disintegrating Agents

Disintegrating agent may be used in the dosage form. Examples of disintegrating agents that can be used are for instance cross-linked polyvinyl pyrrolidone, pregelatinized starch, microcrystalline cellulose, and cross-linked sodium carboxymethyl cellulose.

Alkaline Additives

According to one embodiment of the invention, the active substance may also be mixed with an alkaline pharmaceutically acceptable substance (or substances). Such substances can after excluding bicarbonate salts or carbonate salts be chosen among, but are not restricted to, substances such as the sodium, potassium, calcium, magnesium and aluminium salts of phosphoric acid, citric acid or other suitable weak inorganic or organic acids; substances normally used in antacid preparations such as aluminium, calcium and magnesium hydroxides; magnesium oxide; organic pH-buffering substances such as trihydroxymethylamino methane, basic amines or amino acids and their salts or other similar, pharmaceutically acceptable pH-buffering substances.

Separating or Subcoating Layer

The separating or subcoating layer(s) can be applied to the core material by coating or layering procedures in suitable equipments such as coating pan, coating granulator or in a fluidized bed apparatus using water and/or organic solvents for the coating process. As an alternative the separating layer(s) can be applied to the core material by using powder coating technique. The materials for separating layers are pharmaceutically acceptable compounds such as, for instance, sugar, polyethylene glycol, polyvinylpyrrolidone, polyvinyl alcohol, polyvinyl acetate, hydroxypropyl cellulose, methyl-cellulose, ethylcellulose, hydroxypropyl methyl cellulose, carboxymethylcellulose sodium and others, used alone or in mixtures. Additives such as plasticizers, colorants, pigments, fillers, anti-tacking and ant static agents, such as for instance magnesium stearate, titanium dioxide, fumed silica, talc and other additives may also be included into the separating layer(s).

The separating layer(s) may serve as a diffusion barrier and may act as a pH-buffering zone. The pH-buffering properties of the separating layer(s) can be further strengthened by introducing into the layer(s) substances, after excluding bicarbonate salts or carbonate salts, chosen from a group of compounds usually used in antacid formulations such as, for instance, magnesium oxide, hydroxide, aluminium or calcium hydroxide or silicate; composite aluminium/magnesium compounds such as, for instance MgO.Al₂O₃.2SiO₂.nH₂O, or other pharmaceutically acceptable pH-buffering compounds such as, for instance the sodium, potassium, calcium, magnesium and aluminium salts of phosphoric, citric or other suitable, weak inorganic or organic acids; or suitable organic bases, including basic amino acids or amines and salts thereof. Talc or other compounds may be added to increase the thickness of the layer(s) and thereby strengthen the diffusion barrier.

Enteric Coating Layer

One or more enteric coating layers are applied onto the core material or onto the core material covered with separating layer(s) by using a suitable coating technique. The enteric coating layer material may be dispersed or dissolved in either water or in suitable organic solvents. As enteric coating layer polymers one or more, separately or in combination, of the following can be used; e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s).

The enteric coating layers contain pharmaceutically acceptable plasticizers to obtain the desired mechanical properties, such as flexibility and hardness of the enteric coating layers. Such plasticizers are for instance, but not restricted to, triacetin, citric acid esters, phthalic acid esters, dibutyl sebacate, cetyl alcohol, polyethylene glycols, polysorbates or other plasticizers.

Suspension Modifying Granulate

The suspension modifying granulate comprises;

a rapidly dissolving diluent,
a gelling agent,
an acidic pH-regulating agent,
a binder, and
an optional disintegrant
   and in addition it is free from bicarbonate salts or carbonate salts, especially components that can result in effervescense.

According to one embodiment, the suspension modifying granulate is manufactured by a process in which the rapidly dissolving diluent and the gelling agent are mixed and granulated together, and thereafter dried.

The final moisture content in the suspension modifying granulate measured as loss on drying is <3% (w/w), preferably <1% (w/w). The final content of ethanol is <0.2% (w/w), preferably less than 0.12% (w/w).

When the suspension modifying granulate is suspended in tap water, a stable and close to maximum viscosity is obtained in a short time. Further, the suspension obtained is free from lumps and is robust, in the sense that its viscosity properties are approximately the same even if a patient adds too little or too much water when preparing the suspension-from the granulate. Thus, it is possible to add one dose of the active ingredient and the suspension modifying granulate to from 50% up to 150% of the prescribed amount of water and still obtain the desired properties of the formulation.

The gel formed when adding the suspension modifying granulate to an aqueous medium, such as water, has a viscosity of 3.0 to 6.0 log (mPas)=10³ to 10⁶ mPas, preferably 3.6 to 4.7 log (mPas)=10^{3.6} to 10^{4.7} mPas.

This viscosity is evaluated at 20° C. from the intercept at the viscosity axis of the line when plotting log (viscocity) against log (rotational speed (rpm)). The line is made by a linear fit using least square linear regression and the intercept of the fitted line is determined. Suitable equipment for determination of viscosity is used, such as a Physica DV-1 P viscometer, measuring geometry is No. 2 spindle, 18.7 mm in diameter, length 6.9 mm, which is operated at rotational speed 3.0, 6.0, 30 and 100 rpm, and measurements are made until a stable value is obtained (about 1 minute).

The rapid disintegration and quick gelling to a stable and reproducible viscosity level when the suspension modifying granulate is suspended in water is achieved, according to one aspect of the invention when a special manufacturing process for preparing the suspension modifying granulate is used.

This manufacturing process including the following steps in the following order not excluding the alternative that steps I and II can be interchanged;

I) mixing the gelling agent with the pH-regulating agent, the rapidly dissolving diluent and the optional disintegrant

II) dissolving the binder in ethanol

III) wetting the mixture obtained in step I (alternatively in step II if the order is interchanged) with the solution obtained in step II (alternatively in step I if the order is interchanged)

IV) agitating the wet mixture obtained in step III in order to have almost each particle of the gelling agent to be in close/intimate contact with the above mentioned rapidly dissolving diluent

V) drying the agitated wet mixture from step IV until the final moisture content in the suspension modifying granulate measured as loss on drying is <3% (w/w), preferably <1% (w/w)

VI) grinding or milling the dry granules obtained in step V until more than 95% (w/w) of the granules passes a sieve having 1.0 mm openings.

One feature of the invention is to bring the rapidly dissolving diluent into close/intimate contact with the gelling agent, thereby not only giving a very rapid gelling time compared to the gelling agent per se, but also very quickly a stable gel. One embodiment of the invention is the selection of a suitable rapidly disintegrating diluent, which also may function as a sweetener.

The rapid gelling of the dry suspension modifying granulate in general when added to water, such as tap water, is seen as that gelling is generally sufficient, i.e. reaching 75% of the maximum obtainable level, already within approx. 10 minutes. 90% or more of the maximum viscosity is generally reached within 15 minutes. For comparison see the Table in Example 2.

Specifically, when a single suspension modifying granulate according to the invention is suspended in water and mildly agitated it gives a suspension that reaches more than 75% of the maximum obtainable viscosity within 13 minutes, preferably more than 75% within 10 minutes, tested with 1 g of suspension modifying granulate added to 5 ml of water. More than 90% of the maximum obtainable viscocity is reached within 30 minutes, preferably more than 90% within 25 minutes, tested with 1 g of suspension modifying granulate added to 5 ml of water.

According to one embodiment of the invention the suspension modifying granulate (and the enteric coated PPI comprising pellets) does not contain lactose.

Gelling Agent

The gelling agent provides for forming a gel suitable for administration through a gastric sond/naso-gastric tube, i.e. chosen to have the proper viscoelasticity as well as the proper viscosity of the gel formed when dispersed in an aqueous medium, such as water. This is a desired administration route in pediatric or geriatric therapy.

The dissolution time will also influence the selection of gelling agents.

Suitable gelling agents of the invention are different qualities of xanthan gums.

Also other gelling agent can be considered, but in the case of e.g. certain starch products the suitable range of concentrations are very limited, e.g. Thick-It™ regular, containing modified corn starch and maltodextrin. This product should be used only in the narrow range of about 6 to 8% of the final suspension, corresponding to a content of gelling agent in the suspension modifying granulate of 34 to 48%, which is an unsuitably high proportion of the composition.

Another example is corn starch, that will many times give a rapid swelling, but has undesired viscoelastic properties.

Gelling agents like Na carboxymethylcellulose (CMC) and carrageenan could not be used in the present invention due to lack of suitable viscoelastic properties or due to unsuitable properties for administering the obtained suspension through a gastric sond.

Thus, gelling agents in the invention are chosen among xanthan gums.

The concentration of the gelling agent is 0.6 to 12% w/w of the suspension modifying granulate. In a preferred embodiment the concentration of the gelling agent is between 1.8 to 4.8% w/w. It is suitable to have such a broad range in concentration of the gelling agent of practical reasons for the patient and still having suitable properties of the viscoelestic gel.

In one embodiment of the invention the manufacturing is performed with the gelling agent having an average particle size larger than 150 microns.

Rapidly Dissolving Diluent

The diluent has a diluting function but it may also function as a sweetener.

The diluent is selected from the group consisting of mono- and disaccharides and hydrates of either of them. According to one aspect of the invention preferred diluents are glucose or sucrose or hydrates of either of them. With rapid dissolution is ment, according to the present invention, that the dissolution time of the diluent is below 2 minutes when 2 g of the substance is dissolved in 10 ml of water during slow continuos stirring at 14° C. One diluent specifically not fullfilling this requirement is mannitol.

As a consequence of the manufacturing method, the suspension modifying granulate according to the invention has the rapidly dissolving diluent randomly distributed in and on the obtained individual granule particles.

Acidic pH-Regulating Agent

The suspension modifying granulate when suspended in water forms a suspension having a pH in the range from 3.0 to 6.0, preferably in the range from 3.0 to 5.0, and more preferably in the range from 3.5 to 4.5.

This may be achieved by adding a suitable acidic pH-regulating agent. This agent may consist of a single acidic chemical compound or a mixture of compounds chosen among acidic and alkaline compounds, with the exception of any carbonate salts. Any mixture of such pH influencing compounds is chosen in such a way that when the mixture is dissolved/suspended in water it will give a pH within the desired (acidic) range according to above.

Non-limiting examples of suitable acidic compounds are; Citric acid, tartaric acid and malic acid. Non limiting example of a mixture of compounds chosen among acidic and alkaline compounds is monosodiumphosphate and disodium phosphate (in appropriate ratio to achieve a pH within the desired range).

Disintegrant

The optional disintegrant used in the dry suspension modifying granulate may be a single disintegrant or a mixture thereof.

Non-limiting example of suitable disintegrants include: Polyvinyl pyrrolidone cross-linked, crosslinked Sodium carboxymethyl cellulose (Ac-Di-Sol®) and pregelatinized starch (Sta-Rx® 1500).

Binder

A suitable binder used according to the present invention is a polymer that is soluble in water and in ethanol. Suitable ones are selected qualities of hydroxypropylcellulose. When the binder is chosen to be a hydroxypropyl cellulose (in the following also referred to as HPC), it is having a hydroxypropyl content in the range of 50-90%, or more preferably in the range of 60-81%, and a viscosity below 450 mPas (cps) tested at 5% concentration. Such a polymers are, example given, Klucel® JF and Klucel® LF from Aqualon.

The hydroxypropyl celluloses contemplated for use in this aspect of the invention, as a binder, do not include Low-substituted hydroxypropyl cellulose, also referred to as L-HPC.

The ratio between the binder and the gelling agent in the suspension modifying granulate of the invention is preferably from 1:2 to 1:3, w/w.

Dosage Form Strengths

Different product strengths are obtained by filling specific amounts of enteric coated proton pump inhibitor pellets and the suspension modifying granulate of the invention into unit size sachets. According to one embodiment of the invention the enteric coated pellets comprising esomeprazole magnesium trihydrate are filled together with the suspension modifying granulate into unit size sachets.

The ratio w/w between the two components of the mixture—i.e. between the proton pump inhibitor comprising enteric coated pellets on the one hand and the (dry) suspension modifying granulate on the other hand—may vary between 1:1000 to 100:1000, preferably between 4:1000 to 80:1000 and most preferably between 8:1000 to 60:1000.

Amount of Enteric Coated Pellets in One Sachet.

The PPI comprising enteric coated pellets have a drug content from 5% w/w of the enteric coated pellets to 40% w/w of the enteric coated pellets. This means that the highest theoretical amount of pellets for one dose can be calculated considering the situation when the lowest concentration of drug in pellets is at hand for the highest dose of drug (100 mg acc. to invention) giving a total of (100/0.05=) 2000 mg pellets.

The lowest possible amount of pellets can in an analogue reasoning for the opposite situation be calculated from the highest concentration and the lowest dose (1 mg acc. to the invention) gives the minimum amount of pellets to (1/0.4=) 2.5 mg pellets.

In a preferred embodiment of the invention the drug content of the enteric coated pellets is 8-30% w/w.

The amount of enteric coated pellets in one sachet according to the invention is in the range of 2.5-2000 mg, and in the preferred embodiment of the invention the amount of enteric coated pellets in one sachet is in the range of 3-1250 mg.

In an alternative embodiment of the invention, the drug content of the enteric coated pellets are adapted to the dose of drug intended in one sachet, according to the following table;

TABLE 1

| Dose in one | Adapted drug content in the | Amount of pellets in one |
|---|---|---|
| sachet intended | enteric coated pellets | sachet |
| 1 mg-40 mg | 8-12% w/w | 8-500 mg |
| >40 mg-70 mg | 15-25% w/w | 160-467 mg |
| >70 mg-100 mg | 25-40% w/w | 280-400 mg |

Thus in one embodiment of the invention the dose in one sachet is 1-40 mg and the drug content in the enteric coated pellets is 8-12% (w/w).

In another embodiment of the invention the dose in one sachet is >40 mg-70 mg and the drug content in the enteric coated pellets is 15-25% (w/w).

In a further embodiment of the invention the dose in one sachet is >70 mg-100 mg and the drug content in the enteric coated pellets is 25-40% (w/w).

Liquid Formulation Ready for Use

Prior to use the content of the sachet is emptied into a predefined volume of aqueous liquid. After stirring, a viscous suspension is formed. This liquid formulation is another aspect of the invention and is comprising three main components being enteric coated pellets comprising proton pump inhibitor, (dry) suspension modifying granulate and aqueous liquid.

The amount of aqueous liquid is aimed to be 5 times the amount of suspension modifying granulate, but allowing for the patient to vary this liquid amount from 50% up to 150% of the prescribed amount. This means that the aqueous liquid amount in the liquid formulation ready for use is from 2.5 times up to 7.5 times the amount of the suspension modifying granulate.

In one aspect of the invention the aqueous liquid is water.

The concentration of the gelling agent should be 0.1 to 2% w/w. (a twenty fold range in concentration) of the suspension, preferably between 0.3 to 0.8% w/w. It is advantages to have such a broad range in concentration of the gelling agent of practical reasons for the patient and still maintaining relevant properties of the viscoelestic gel.

### EXAMPLES

### Example 1a

### Preparation of the Suspension Modifying Granulate According to the Invention

| Excipient | Content (%) |
|---|---|
| Xanthan Gum 11K | 2.5 |
| Polyvinylpyrrolidone cross-linked | 2.5 |
| Glucose, water free | 93.8 |
| Hydroxypropyl cellulose JF | 1.0 |
| Citric acid anhydrous | 0.164 |
| Colour iron dioxide yellow | 0.06 |

The hydroxypropyl cellulose is dissolved in ethanol. The solution is added to a dry mixture of the remaining excipients giving a wet mass and the mass being granulated during the addition of the solution. The wet mass is dried and grinded (maximum 5% of the granules >1 mM).

3 g of this suspension modifying granulate was dissolved in 15 ml water and the liquid formulation was stirred for 60 s. The pH was measured with a glass electrod using a calibrated pH meter and found to be 4.0.

### (Comparative) Example 1b

### Suspension Modifying Granulate According to Prior Art

As comparison has been used the commercially product “Lanzo™ 30 mg, granulate” from Wyeth Lederle (batch 3ET032, expiry date July 2006 and 3ET010, expiry date March 2006.

The suspension granulate composition (excluding the enteric coated pellets) used for this product is according to SWEDIS:

| Excipient | Content (%) |
|---|---|
| Mannitol | 45.8 |
| Sucrose | 45.8 |
| Xanthan gum | 3.5 |
| Polyvinylpyrrolidone, cross-linked | 3.5 |
| Dioctyl sulfosuccinate | 0.015 |
| Magnesium stearate | 0.5 |
| Silicon dioxide | 0.1 |
| Citric acid anhydrous | 0.4 |
| Color | 0.05 |
| Flavouring | 0.4 |

### Ex 2

### Viscosity Measurements

Experimental Conditions:

Embodiment according to the invention: 3 g suspension modifying granulate obtained according to Example 1a was dissolved in 15 ml water and the liquid formulation was stirred for 60 s.

Prior art sample (Lanzo™ 30 mg, granulate): The Lansoprazole comprising pellets were removed from the total solids (5.7 g) of the product described in Example 1b and to the remaining powder/granulate (5.4 g) was added 30 ml water whereafter the liquid formulation was stirred for 60 s.

For both samples viscosity measurements started after another 1 min.

Instrument: Reologica Stresstech

Measuring principle: Oscillation with plate/plate P 30 2 mm slit

Measuring parameters: Frequency 0.1 Hz; stress 0.07146 Pa.

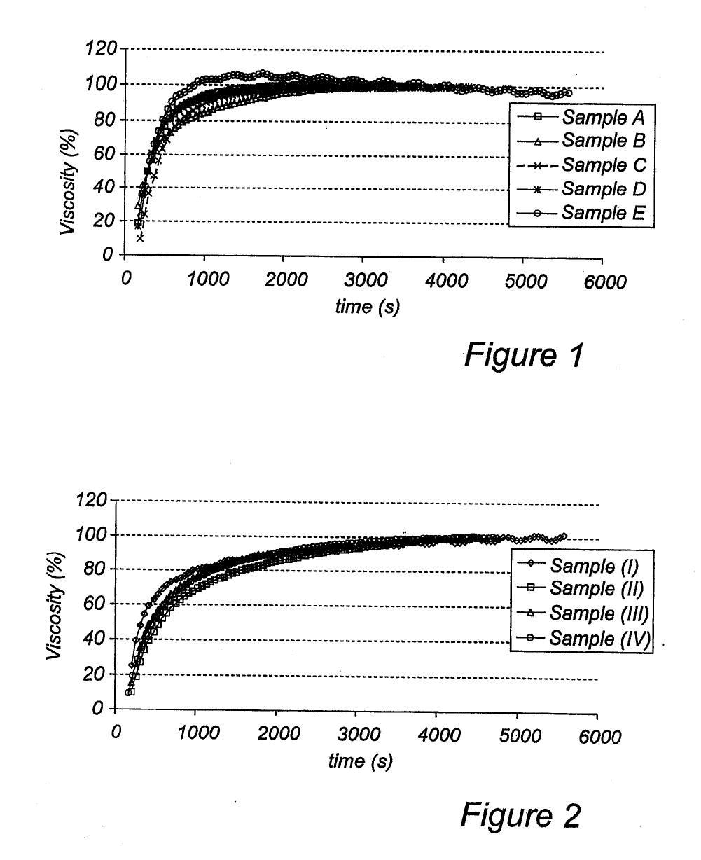

Time to arrive at Viscosity in % of Maximum Viscosity
(Evaluated from FIG. 1 and 2.)

| | Percentage of maximum | |
|---|---|---|
| | Viscosity | |
| | >75% | >90% |
| Ex. 1a (invention) n = 5 | average = 9.7 min | average = 14.8 min |
| | min = 7.6 min | min = 9.5 min |
| | max = 12.6 min | max = 23.1 min |
| Ex. 1b (prior art) n = 4 | average = 16.8 min | average = 32.5 min |
| | min = 13.3 min | min = 29.0 min |
| | max = 21.2 min | max = 39.5 min |

Discussion

In the case of lansoprazole (Ex. 1b), although having a fast dissolving diluent (sucrose) being added to the suspension granulate formulation, the formulation will not form a stable gel within the desired shorter time frame, see FIG. 2, to be compare with that which is obtained with the present invention (Ex. 1a), see FIG. 1, and the results shown in the Table above.

The result of using a slow dissolving diluent will be a composition with slower gelling and a continuous increasing viscosity within a reasonable and adequate time period. Thus, the present invention has solved several problems in order to obtain a lactos-free and bicarbonate/carbonate-free composition having rapid gelling time with a viscosity/viscoelasticity suitable for swallowing or administration through a tube, such as constant viscosity over time, and no lumps present in the final suspension to be administered.

### Example 3

### Manufacturing of Enteric Coated Pellets Comprising Esomeprazole-Mg-Trihydrate

| Core material | | |
|---|---|---|
| Esomeprazole-Mg trihydrate | 445 | g |
| Sugar sphere seeds | 300 | g |
| Hydroxypropyl methylcellulose | 67 | g |
| Polysorbate 80 | 9 | g |
| Purified water | 2100 | g |
| Subcoating layer | | |
| Hydroxypropyl cellulose | 90 | g |
| Talc | 340 | g |
| Magnesium stearate | 22 | g |
| Purified water | 3100 | g |
| Enteric coating layer | | |
| Methacrylic acid copolymer type C, 30% dispersion | 1270 | g |
| Triethyl citrate | 38 | g |
| Mono- and diglycerides | 19 | g |
| Polysorbate 80 | 2 | g |
| Purified water | 500 | g |

Suspension layering was performed in a fluid bed apparatus using bottom spray technique. Esomeprazole was sprayed onto the sugar sphere seeds from a water suspension containing the dissolved binder and surfactant. The size of the sugar spheres seeds were in the range of 0.25 to 0.35 mm.

The prepared core material was covered with the subcoating layer in a fluid bed apparatus with a hydroxypropyl cellulose solution containing talc and magnesium stearate. The enteric coating layer was sprayed as a water dispersion onto the pellets covered with separating layer in a fluid bed apparatus.

### Example 4

### Examples of Component Ratios for Preparing Final Liquid Formulation of Different Dose Strength

| | Strength (amount of active | | |
|---|---|---|---|
| | drug, e.g.esomeprazole, | | |
| | per sachet) | | |
| | 2.5 mg | 10 mg | 40 mg |
| Amount of enteric coated. pellets*, | 10.6 | 42.6 | 170 |
| (in sachet) (mg) | | | |
| Amount of suspension modifying | 1 | 3 | 3 |
| granulate** (in same sachet as above) (g) | | | |
| Volume of water (ml) | 5 | 15 | 15 |

*made in accordance with Example 3.
**made in accordance with Example 1a.

### Example 5

### Illustration of the Rapid Gelling Time of the Present Invention

The content of a sachet containing the final formulation, of 40 mg dose strength according to Ex. 4 was emptied into a beaker containing the nominally prescribed 15 ml amount of water.

Then the sample was stirred for 15 seconds and then kept resting until 55 seconds from start. After that it was again stirred for 5 seconds, to evenly distribute the active drug granules in the suspension.

Then the suspension was inspected for 30 seconds to determine whether substantially all of the enteric coated pellets were distributed in the suspension or if they were assembled at the bottom of the beaker.

If the pellets were not distributed in the liquid medium but assembled at the bottom of the beaker, the process was repeated, i.e. meaning waiting 25 seconds further and stirring 5 seconds, i.e. until 2 minutes, followed by inspecting for 30 seconds, until substantially all of the pellets remained distributed in the liquid medium. The time needed for the pellets to be remaining in the liquid medium was recorded.

The samples in the table below were evaluated in the described way, with the following results;

| | Time needed |
|---|---|
| | for pellets |
| Sample | to remain suspended |
| 1) Sachet cont. 40 mg dose strength, acc. to Ex. 4. | 2 minutes |
| 2) Sachet cont. 40 mg dose strength, acc. to Ex. 4. | 2 minutes |
| 2) Sachet cont. 10 mg dose strength, acc. to Ex. 4. | 2 minutes |
| I) Sachet “Lanzo ™ 30 mg” | 5 minutes |
| II) Sachet “Lanzo ™ 30 mg” | 5 minutes |

1-32. (canceled)

33. A solid oral pharmaceutical dosage form comprising:
(a) a multitude of enteric coated pellets, wherein each enteric coated pellet comprises an acid sensitive proton pump inhibitor, and (b) a suspension modifying granulate comprising a rapidly dissolving diluent, a gelling agent which is a xanthan gum, an acidic pH-regulating agent, a binder, and optionally, a disintegrant,
wherein the dosage form is a rapidly-gelling granulate mixture,
with the proviso that the granulate is free from bicarbonate and carbonate salts.

34. The dosage form according to claim 33, which is free from lactose.

35. The dosage form according to claim 33, wherein the suspension modifying granulate is obtained by mixing and granulating the rapidly dissolving diluent and the gelling agent together such that the diluent is randomly distributed throughout the obtained granulate particles.

36. The dosage form according claim 33, wherein the concentration of the gelling agent is 0.6% to 12% (w/w) of the suspension modifying granulate.

37. The dosage form according claim 33, wherein the concentration of the gelling agent is 1.8% to 4.8% (w/w) of the suspension modifying granulate.

38. The dosage form according to claim 33, wherein the suspension modifying granulate when suspended in water forms a suspension having a pH in the range of between 3.0 and 6.0.

39. The dosage form according to claim 33, wherein the suspension modifying granulate when suspended in water forms a suspension having a pH in the range of between 3.0 and 5.0.

40. The dosage form according to claim 33, wherein the ratio of the binder to the gelling agent in the suspension modifying granulate is in the range of from 1:2 to 1:3 (w/w).

41. The dosage form according to claim 33, wherein the rapidly dissolving diluent is selected from the group consisting of monosaccharides, hydrates of monosaccharides, disaccharides, and hydrates of disaccharides.

42. The dosage form according to claim 41, wherein the rapidly dissolving diluent is selected from the group consisting of glucose, hydrates of glucose, sucrose, and hydrates of sucrose.

43. The dosage form according to claim 33, wherein the proton pump inhibitor is omeprazole or a magnesium salt of omeprazole.

44. The dosage form according to claim 33, wherein the proton pump inhibitor is esomeprazole, an alkaline salt of esomeprazole, a hydrate of esomeprazole, or a hydrate of the alkaline salt of esomeprazole.

45. The dosage form according to claim 33, wherein the proton pump inhibitor is tenatoprazole, a pharmaceutically acceptable salt of tenatoprazole, a single enantiomer of tenatoprazole, or a pharmaceutically acceptable salt of the single enantiomer.

46. The dosage form according to claim 33, wherein the enteric coated pellets comprise a core material, a subcoating layer, and an enteric coating layer, with the proviso that the pellets do not have an additional coating layer on the enteric coating layer.

47. The dosage form according to claim 33, wherein the enteric coated pellets have an average diameter in the range of 0.2-1.8 mm.

48. The dosage form according to claim 33, wherein the enteric coated pellets have an average diameter in the range of 0.4-1.0 mm.

49. The dosage form according to claim 33, wherein the suspension modifying granulate, when suspended and agitated in an aqueous liquid, gives a suspension having, within 13 minutes, a viscosity which is at least 75% of the maximum obtainable viscosity.

50. The dosage form according to claim 33, wherein the suspension modifying granulate, when suspended and agitated in an aqueous liquid, gives a suspension having, within 10 minutes, a viscosity which is at least 75% of the maximum obtainable viscosity.

51. The dosage form according to claim 33, wherein the suspension modifying granulate, when suspended and agitated in an aqueous liquid, gives a suspension having, within 30 minutes, a viscosity which is at least 90% of the maximum obtainable viscosity.

52. The dosage form according to claim 33, wherein the suspension modifying granulate, when suspended and agitated in an aqueous liquid, gives a suspension having, within 25 minutes, a viscosity which is at least 90% of the maximum obtainable viscosity.

53. A sachet comprising the dosage form according to claim 33.

54. The sachet according to claim 53, wherein the amount of the proton pump inhibitor in the dosage form is in the range of 1 mg-100 mg.

55. The sachet according to claim 53, wherein the amount of the proton pump inhibitor in the dosage form is in the range of 1 mg-40 mg.

56. A ready-for-use liquid formulation comprising an aqueous liquid and the dosage form according to claim 33.

57. The liquid formulation according to claim 56, wherein the amount of the aqueous liquid is in the range of from 2.5 times up to 7.5 times the amount of the granulate.

58. The liquid formulation according to claim 56, wherein the suspension modifying granulate, when suspended and agitated in the aqueous liquid, gives a suspension having, within 13 minutes, a viscosity which is at least 75% of the maximum obtainable viscosity.

59. The liquid formulation according to claim 56, wherein the suspension modifying granulate, when suspended and agitated in the aqueous liquid, gives a suspension having, within 10 minutes, a viscosity which is at least 75% of the maximum obtainable viscosity.

60. The liquid formulation according to claim 56, wherein the suspension modifying granulate, when suspended and agitated in the aqueous liquid, gives a suspension having, within 30 minutes, a viscosity which is at least 90% of the maximum obtainable viscosity.

61. The liquid formulation according to claim 56, wherein the suspension modifying granulate, when suspended and agitated in the aqueous liquid, gives a suspension having, within 25 minutes, a viscosity which is at least 90% of the maximum obtainable viscosity.

62. The liquid formulation according to claim 56, wherein the aqueous liquid is water.

63. A process for preparing the suspension modifying granulate used in the dosage form according to claim 33, wherein the process comprises mixing together and granulating the rapidly dissolving diluent and the gelling agent together, and subsequently drying the obtained suspension modifying granulate, wherein the diluent is randomly distributed throughout the obtained individual granulate particles.

64. A process for preparing the suspension modifying granulate used in the dosage form according to claim 33, wherein the process comprises the steps of:
I) mixing the gelling agent with the pH-regulating agent, the rapidly dissolving diluent, and the optional disintegrant;
II) dissolving the binder in ethanol;
III) wetting the mixture obtained in step I with the solution obtained in step II;
IV) agitating the wet mixture obtained in step III such that substantially each particle of the gelling agent is in contact with the rapidly dissolving diluent;
V) drying the agitated wet mixture from step IV until the final moisture content in the granulate measured as loss on drying is less than 3% (w/w); and
VI) grinding or milling the dry granules obtained in step V until more than 95% (w/w) of the granules pass through a sieve having 1.0 mm openings.

65. The process of claim 64, wherein step II is performed before step I.

66. The method according to claim 64, wherein the loss on the final moisture content in the granulate measured as loss on drying is less than 1% (w/w).

67. A method of treatment of gastric acid related diseases comprising administering an effective amount of an oral pharmaceutical dosage form as defined in claim 33 to a patient in need thereof.

68. The method according to claim 67, wherein the patient is a child or elderly.
